(19) United States
(12) Patent Application Publication
(10) Pub. No.: US 2024/0115402 A1
(43) Pub. Date: Apr. 11, 2024

(54) MAMMARY PROSTHESIS AND ARTIFICIAL NIPPLE

(71) Applicant: Natural Breast Co,. Ltd., Fukuoka (JP)

(72) Inventor: Yukie HONDA, Fukuoka (JP)

(73) Assignee: Natural Breast Co,. Ltd., Fukuoka (JP)

(21) Appl. No.: 18/276,890

(22) PCT Filed: Feb. 15, 2021
(86) PCT No.: PCT/JP2021/005476

§ 371 Date: Aug. 11, 2023

(51) Int. Cl. A61F 2/52

(52) U.S. Cl. CPC A61F 2/52; A61F 2002/523; A61F 2002/526

(57) ABSTRACT

A mammary prosthesis and an artificial nipple are provided. This mammary prosthesis includes a first layer, a second layer, and an outer layer. The first layer is constituted by including a mixture of a silicone rubber and at least two thickening agents having mutually different viscosities. A hollow is formed inside the first layer. The second layer is a layer harder than the first layer, and is laminated on the outer portion of the first layer in a state of covering the entire first layer. The outer layer is laminated on the curved portion of the second layer opposite the first layer. The outer layer is constituted by a colored layer, a cover layer, and a reflection-suppressing layer. The cover layer is disposed outside the colored layer, and the reflection-suppressing layer is disposed outside the cover layer.

### BACKGROUND

### Technical Field

The present invention relates to a mammary prosthesis and an artificial nipple. In detail, the present invention relates to a mammary prosthesis and an artificial nipple for a woman who has lost her breast, for example.

### Description of Related Art

For a woman, a deformation or loss of her breast has a significant psychological impact. As in need of a shape control pad when putting on underwear, etc., her daily activities are hindered and the quality of life is lowered.

Thus, in order to improve the quality of life, a breast reconstruction surgery is performed as a method of returning the deformed or lost breast to as normal a state as possible. However, the breast reconstruction surgery has disadvantages such as an occurrence of infections or skin necrosis.

Meanwhile, there are many women who dislike such disadvantages and do not undergo the breast reconstruction surgery and instead attach a mammary prosthesis which is an artificial object to the skin of the chest portion, and various mammary prosthesiss have been proposed.

For example, in Patent Literature 1, a mammary prosthesis as shown in FIG. 5 is described. That is, a mammary prosthesis 101 described in Patent Literature 1 is configured by a bowl-shaped outer skin portion 102 which is the surface side, and a bottom skin portion (bottom surface portion) 103. In addition, an internal space 104 is formed between the outer skin portion 102 and the bottom skin portion 103, and the mammary prosthesis 101 is formed in a hollow bag shape as a whole.

In addition, the outer skin portion 102 and the bottom skin portion 103 are made of soft resin such as silicone rubber for medical use, and a surface of the outer skin portion 102 is colored to match the color of the chest portion of a user who is a wearer.

A papilla portion 105 is provided in the vicinity of the center of the outer skin portion 102. In addition, in the vicinity of the center of the bottom skin portion 103, a concave portion 106 is formed to be recessed to the outer skin portion 102 side, and the internal space 104 is formed between the concave portion 106 and the outer skin portion 102. In addition, a needle insertion valve 107 is formed in a central portion of the concave portion 106 so that air pressure of the internal space 104 can be adjusted by letting air into and out from the internal space 104.

### CITATION LIST

### Patent Documents

Patent Document 1: Japanese Unexamined Patent Application Publication No. 2016-209340

### SUMMARY

### Technical Problems

In the meantime, since the mammary prosthesis is directly put on a human body and used daily, silicone rubber of the mammary prosthesis easily deteriorates and becomes unnaturally hard in comparison to a human breast.

The present invention is made in consideration of the point described above, and an object of the present invention is to provide a mammary prosthesis that is less likely to become unnatural in comparison to a human breast, and an artificial nipple that is less likely to become unnatural in comparison to a human nipple.

### Solution to Problems

In order to achieve the object described above, a mammary prosthesis of the present invention includes a first layer configured by containing a mixture of silicone rubber and at least two types of thickeners whose viscosities are different from each other, the first layer inside which a hollow space is formed, and a second layer arranged on the outer side of the first layer, the second layer being harder than the first layer, the second layer being configured by containing silicone rubber.

With the first layer configured by containing the mixture of silicone rubber and at least two types of thickeners whose viscosities are different from each other, the mammary prosthesis is softer than a mammary prosthesis not containing such thickeners, and it is possible to keep its softness for a long time.

In addition, with the second layer arranged on the outer side of the first layer, the second layer being harder than the first layer, it is possible to adjust a shape of the mammary prosthesis.

In addition, with the first layer configured by containing the mixture of silicone rubber and at least two types of thickeners whose viscosities are different from each other and the second layer arranged on the outer side of the first layer, the second layer being harder than the first layer, the mammary prosthesis becomes harder stepwise from the inner side to the outer side. Thus, even when a force is applied from an exterior, unnatural wrinkles are not easily made.

In addition, the mammary prosthesis of the present invention can have a configuration of including a colored layer arranged on the outer side of the second layer, the colored layer being configured by containing a colorant, and a cover layer arranged on the outer side of the colored layer, the cover layer being capable of suppressing peeling of the colored layer.

In this case, with the colored layer arranged on the outer side of the second layer, the colored layer being configured by containing the colorant, it is possible to perform external coloring and bring the texture closer to the texture of the human skin than in the case of internal coloring where the first layer or the second layer contains a colorant.

In addition, with the cover layer arranged on the outer side of the colored layer, the cover layer being capable of suppressing the peeling of the colored layer, it is possible to maintain the texture brought close to the texture of the human skin.

Further, the mammary prosthesis of the present invention can have a configuration that the colored layer has a nipple region in which a nipple is displayed, and the nipple region and other regions of the colored layer are positioned in substantially the same plane.

In this case, it is possible to make the nipple inconspicuous even when the nipple is seen through clothing.

In addition, the mammary prosthesis of the present invention can have a configuration of including a reflection suppression layer arranged on the outer side of the cover layer, the reflection suppression layer being capable of suppressing reflection of light of the cover layer or the colored layer.

In this case, it is possible to bring the texture even closer to the texture of the human skin.

In addition, the mammary prosthesis of the present invention can have a configuration of including a thin film layer arranged on the outer side of the second layer, the thin film layer being capable of blocking a substance, with the thin film layer being transparent.

In this case, the thin film layer can block a substance. Thus, it is possible to prevent a substance from entering the first layer and the second layer from the exterior of the mammary prosthesis, and to prevent a substance contained in the first layer or the second layer from leaking out to the exterior of the mammary prosthesis. In addition, since the thin film layer is transparent, it does not feel strange.

In addition, the mammary prosthesis of the present invention can have a configuration of including an adhesive layer arranged attachably to and detachably from a layer which is arranged on the outermost side, the adhesive layer being configured by containing an adhesive material. In this case, it is possible to improve an adhesive force of the mammary prosthesis with respect to the human body.

In addition, the mammary prosthesis of the present invention can have a configuration that a through hole passing through the first layer and the second layer and providing communication between an exterior and the hollow space, the through hole having such a size that a gas or a liquid in the hollow space is not easily leaked out to the exterior is formed in the first layer and the second layer.

In this case, through the through hole, it is possible to introduce a gas or a liquid into the hollow space formed inside the first layer from the exterior of the mammary prosthesis of the present invention and retain the introduced gas or liquid in the hollow space.

In addition, in order to achieve the object described above, a mammary prosthesis of the present invention includes a silicone rubber layer configured by containing silicone rubber, the silicone rubber layer inside which a hollow space is formed, a nipple portion provided in part of the silicone rubber layer on the opposite side of the hollow space, the nipple portion being configured by containing a mixture of silicone rubber and at least two types of thickeners whose viscosities are different from each other, a colored layer arranged on the outer side of the silicone rubber layer and on the outer side of the nipple portion, the colored layer being configured by containing a colorant, a cover layer arranged on the outer side of the colored layer, the cover layer being capable of suppressing peeling of the colored layer, and a reflection suppression layer arranged on the outer side of the cover layer, the reflection suppression layer being capable of suppressing reflection of light of the cover layer or the colored layer.

With the nipple portion configured by containing the mixture of silicone rubber and at least two types of thickeners whose viscosities are different from each other, the nipple portion is softer than a nipple portion not containing such thickeners, and it is possible to keep its softness for a long time.

In addition, with the colored layer arranged on the outer side of the silicone rubber layer and on the outer side of the nipple portion, the colored layer being configured by containing the colorant, it is possible to perform external coloring and bring the texture closer to the texture of the human skin than in the case of internal coloring where silicone rubber contains a colorant.

In addition, with the cover layer arranged on the outer side of the colored layer, the cover layer being capable of suppressing the peeling of the colored layer, the texture brought close to the texture of the human skin is easily maintained.

In addition, with the reflection suppression layer arranged on the outer side of the cover layer, the reflection suppression layer being capable of suppressing the reflection of light of the cover layer or the colored layer, it is possible to bring the texture even closer to the texture of the human skin.

In addition, in order to achieve the object described above, an artificial nipple of the present invention includes a main body layer configured by containing a mixture of silicone rubber and at least two types of thickeners whose viscosities are different from each other, a colored layer arranged on the outer side of the main body layer, the colored layer being configured by containing a colorant, a cover layer arranged on the outer side of the colored layer, the cover layer being capable of suppressing peeling of the colored layer, and a reflection suppression layer arranged on the outer side of the cover layer, the reflection suppression layer being capable of suppressing reflection of light of the cover layer or the colored layer.

With the main body layer configured by containing the mixture of silicone rubber and at least two types of thickeners whose viscosities are different from each other, the artificial nipple is softer than an artificial nipple not containing such thickeners, and it is possible to keep its softness for a long time.

In addition, with the colored layer arranged on the outer side of the main body layer, the colored layer being configured by containing the colorant, it is possible to perform external coloring and bring the texture closer to the texture of the human skin than in the case of internal coloring where silicone rubber contains a colorant.

In addition, with the cover layer arranged on the outer side of the colored layer, the cover layer being capable of suppressing the peeling of the colored layer, it is possible to maintain the texture brought close to the texture of the human skin.

In addition, with the reflection suppression layer arranged on the outer side of the cover layer, the reflection suppression layer being capable of suppressing the reflection of light of the cover layer or the colored layer, it is possible to bring the texture even closer to the texture of the human skin.

### Advantageous Effects of Invention

The mammary prosthesis according to the present invention is less likely to become unnatural in comparison to a human breast. The artificial nipple according to the present invention is less likely to become unnatural in comparison to a human nipple.

### BRIEF DESCRIPTION OF THE DRAWINGS

FIG. 1 is a schematic sectional view showing a first embodiment of a mammary prosthesis to which the present invention is applied.

FIG. 2(a) is a schematic plan view of a second embodiment of the mammary prosthesis to which the present invention is applied; and FIG. 2(b) is a schematic partial sectional view of the vicinity of a nipple region when the mammary prosthesis shown in FIG. 2(a) is cut along line A-A.

FIG. 3 is a schematic sectional view showing an example of an artificial nipple to which the present invention is applied.

FIG. 4 is a schematic sectional view of the mammary prosthesis of the first embodiment to which the present invention is applied, the mammary prosthesis being put on a chest portion of a human body.

FIG. 5 is a schematic sectional view of a conventional mammary prosthesis.

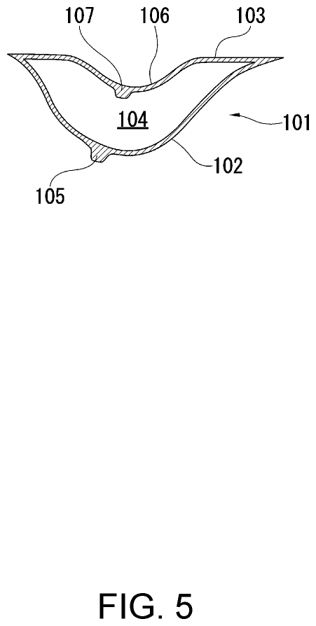

### DESCRIPTION OF THE EMBODIMENTS

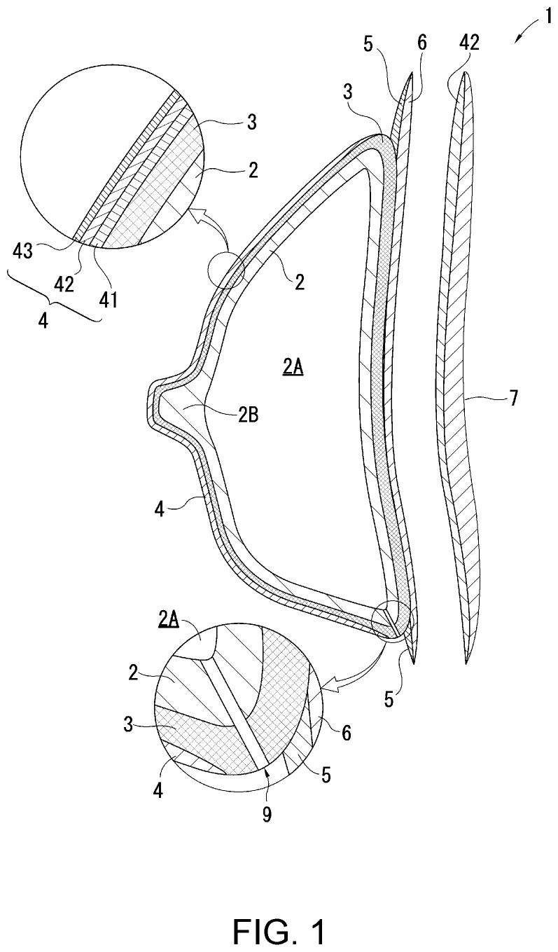

Hereinafter, embodiments of the present invention will be described with reference to the attached drawings in order to facilitate understanding of the present invention. FIG. 1 is a schematic sectional view showing a first embodiment of a mammary prosthesis to which the present invention is applied.

A mammary prosthesis 1 of the present invention shown in FIG. 1 includes a first layer 2. The first layer 2 is configured by containing a mixture of silicone rubber and at least two types of thickeners whose viscosities are different from each other. In addition, a hollow space 2A is formed inside the first layer 2. In addition, softness of the first layer 2 having such a configuration is softer than softness of a silicone rubber layer not containing such thickeners, and the softness is kept at least for 2 years.

In addition, a protruding nipple portion 2B is integrally provided in part of a curved part of the first layer 2 on the opposite side of the hollow space 2A. However, it is also possible to attach a nipple portion to the first layer 2 as a separate body.

In addition, various silicone rubbers can be used as the silicone rubber used for the first layer 2. However, specifically, an example includes silicone rubber having a viscosity of 1,500 to 6,000 mPa·s at a normal temperature.

In addition, the thickeners used for the first layer 2 are not particularly limited as long as the thickeners are at least two types of thickeners whose viscosities are different from each other. In addition, as such thickeners, specifically, an example includes at least two types of thickeners whose viscosities are different from each other selected from a group consisting of thickeners which have a viscosity of 100 to 2,500 mPa·s at a normal temperature.

In addition, when the first layer 2 is configured by containing the mixture of silicone rubber and at least two types of thickeners whose viscosities are different from each other, it is possible to further mix another rubber such as urethane rubber.

In addition, the mammary prosthesis 1 of the present invention includes a second layer 3. The second layer 3 is configured by containing silicone rubber. In addition, the second layer 3 is arranged on the outer side of the first layer 2 and is a layer harder than the first layer 2. That is, the second layer 3 is laminated on an outer side part of the first layer 2 in a state of covering the entire first layer 2.

In addition, as the silicone rubber used for the second layer 3, any silicone rubber can be used as long as the silicone rubber is harder than the silicone rubber used for the first layer 2.

In addition, the mammary prosthesis 1 of the present invention includes an outer covering layer 4. The outer covering layer 4 is arranged on the outer side of the second layer 3. That is, the outer covering layer 4 is laminated on a curved part of the second layer 3 on the opposite side of the first layer 2. In addition, the curved part of the second layer 3 is a part to be positioned on the front surface side when the mammary prosthesis 1 of the present invention is put on a chest portion of a human body.

In addition, the outer covering layer 4 is configured by a colored layer 41, a cover layer 42, and a reflection suppression layer 43.

The colored layer 41 is configured by containing a colorant, specifically, for example, a pigment. In addition, the colored layer 41 is laminated on the curved part of the second layer 3 on the opposite side of the first layer 2.

In addition, the cover layer 42 is capable of suppressing peeling of the colored layer 41. In addition, the cover layer 42 is arranged on the outer side of the colored layer 41. That is, the cover layer 42 is laminated on the colored layer 41 on the opposite side of the second layer 3.

In addition, the reflection suppression layer 43 is capable of suppressing reflection of light of the cover layer 42 or the colored layer 41 by roughening a surface of the cover layer 42 or the colored layer 41. In addition, the reflection suppression layer 43 is arranged on the outer side of the cover layer 42. That is, the reflection suppression layer 43 is laminated on the cover layer 42 on the opposite side of the colored layer 41.

In addition, the cover layer 42 and the reflection suppression layer 43 do not block a color shown by the colored layer 41. Although the cover layer 42 and the reflection suppression layer 43 are arranged on the outer side of the colored layer 41, it is possible to visually recognize the color shown by the colored layer 41 through the cover layer 42 and the reflection suppression layer 43.

In addition, as shown in FIG. 1, the mammary prosthesis 1 of the present invention has a curved part to be positioned on the front surface side when the mammary prosthesis 1 is put on the chest portion of the human body, and a substantially flat part to be positioned on the back surface side facing the chest portion when the mammary prosthesis 1 is put on the chest portion of the human body. A part where the curved part and the flat part are connected is a peripheral edge portion.

In addition, it is also possible to form a concave portion in the mammary prosthesis of the present invention on the back surface side facing the chest portion when the mammary prosthesis is put on the chest portion of the human body.

In addition, the mammary prosthesis 1 of the present invention includes a thin skin portion 5. The thin skin portion 5 is configured by containing silicone rubber, and is so thin that it becomes transparent and is a soft part. In addition, the thin skin portion 5 is provided on the outer side of the second layer 3 over the entire peripheral edge portion. Therefore, when the mammary prosthesis 1 of the present invention is put on the chest portion of the human body, it is possible to see the color of the human skin through the thin skin portion 5.

The thin skin portion 5 is attached to the second layer 3 as a separate body from the second layer 3. However, the thin skin portion 5 may be provided integrally with the first layer 2 and the second layer 3, or integrally with the second layer 3.

In addition, the mammary prosthesis 1 of the present invention includes a polyethylene sheet layer 6. The polyethylene sheet layer 6 is capable of blocking air, water, a phosphorus substance, etc., and is transparent. In addition, a thickness of the polyethylene sheet layer 6 is specifically, for example, 10 to 20 μm. However, as a matter of course, the present invention is not limited to this thickness. Polyethylene is an example of a material of a thin film layer, and it is possible to use other materials as the thin film layer as long as the materials are capable of blocking a substance and are transparent.

In addition, the polyethylene sheet layer 6 is arranged on the outer side of the second layer 3. That is, the polyethylene sheet layer 6 is laminated on a substantially flat part of the second layer 3 on the opposite side of the first layer 2. In addition, part of the polyethylene sheet layer 6 is also laminated on the thin skin portion 5. The polyethylene sheet layer 6 is an example of the thin film layer.

In addition, it is also possible to arrange the thin film layer such as the polyethylene sheet layer 6, for example, in a state where the thin film layer is sandwiched between the first layer 2 and the second layer 3. In this case, the configuration is that the thin film layer such as the polyethylene sheet layer is arranged to cover the entire first layer. Thus, for example, even when a liquid is charged in the hollow space inside the first layer, it is possible to prevent the inside liquid from oozing from a surface of the mammary prosthesis of the present invention to an exterior by the thin film layer such as the polyethylene sheet layer.

In addition, the mammary prosthesis 1 of the present invention includes an adhesive layer 7. The adhesive layer 7 is configured by containing silicone gel, and arranged attachably to and detachably from the polyethylene sheet layer 6 arranged on the outermost side.

That is, the adhesive layer 7 is laminated attachably to and detachably from the polyethylene sheet layer 6 via the cover layer 42. However, it is also possible to omit the cover layer 42. In addition, it is also possible to laminate the adhesive layer 7 attachably to and detachably from the polyethylene sheet layer 6 via another cover layer configured by a different material from the material configuring the cover layer 42.

The silicone gel is an example of an adhesive material, and it is also possible to use, for example, a mixture of silicone gel and silicone rubber as the adhesive material.

In addition, a through hole 9 is formed in the first layer 2 and the second layer 3 provided in the mammary prosthesis 1 of the present invention. The through hole 9 passes through the first layer 2 and the second layer 3 and provides communication between the exterior of the mammary prosthesis 1 of the present invention and the hollow space 2A inside the first layer 2.

In addition, a size of the through hole 9 is such a size that a gas or a liquid in the hollow space 2A is not easily leaked out to the exterior of the mammary prosthesis 1 of the present invention. The size of the through hole 9 is specifically a length in the direction substantially orthogonal to the passing direction of the through hole 9, and specifically, for example, 0.1 to 1 mm. It is noted that FIG. 1 shows the through hole 9 having such a size that the through hole 9 can be visually recognized for descriptive purpose.

In addition, the through hole 9 has such a size, and is formed in the first layer 2 configured by containing silicone rubber and the second layer 3 configured by containing silicone rubber. Thus, by contraction or expansion of the rubber, the through hole 9 is easily contracted and closed, and easily opened by readily expanding it with a needle, etc.

In addition, the through hole 9 is formed in a relatively thick part among a part where the first layer 2 and the second layer 3 overlap with each other. In the example shown in FIG. 1, the through hole 9 is formed in the peripheral edge portion of the mammary prosthesis 1 of the present invention. However, as a matter of course, a point where the through hole 9 is formed is not limited to the peripheral edge portion. For example, it is also possible to form the through hole 9 passing through the nipple portion 2B, or to make a mass of silicone rubber in the second layer 3 on the side in contact with the first layer 2 to have a thick part, and form the through hole 9 passing through this mass.

In addition, it is possible to insert, for example, a needle of a syringe or a needle of an inflator for a tennis ball into the through hole 9, and introduce a gas or a liquid into the hollow space 2A.

The mammary prosthesis of the present invention does not necessarily have to include the colored layer, and for example, the first layer or the second layer can also contain a colorant.

However, it is preferable that the mammary prosthesis of the present invention includes the colored layer since it is possible to perform external coloring and bring the texture closer to the texture of the human skin than in the case of internal coloring where the first layer or the second layer contains a colorant.

In addition, the mammary prosthesis of the present invention does not necessarily have to include the cover layer. However, it is preferable that the mammary prosthesis of the present invention includes the cover layer since the texture brought close to the texture of the human skin is easily maintained.

In addition, the mammary prosthesis of the present invention does not necessarily have to include the reflection suppression layer. However, it is preferable that the mammary prosthesis of the present invention includes the reflection suppression layer since it is possible to bring the texture even closer to the texture of the human skin.

In addition, the mammary prosthesis of the present invention does not necessarily have to include the polyethylene sheet layer, that is, the thin film layer.

However, it is preferable that the mammary prosthesis of the present invention includes the thin film layer since the thin film layer can block a substance, and thus it is possible to prevent a substance from entering the first layer and the second layer from the exterior of the mammary prosthesis, and to prevent a substance contained in the first layer or the second layer from leaking out to the exterior of the mammary prosthesis. In particular, when a substance such as stratum corneum or phosphorus entering the first layer or the second layer moves to and enters the adhesive layer, an adhesive force of the adhesive layer is decreased. Thus, it is possible to suppress a decrease in the adhesive force of the adhesive layer by the thin film layer.

In addition, the mammary prosthesis of the present invention does not necessarily have to include the adhesive layer as long as the mammary prosthesis of the present invention can be put on the chest portion of the human body. It is also possible to put the mammary prosthesis of the present invention on the chest portion of the human body by using other adhesives. However, it is preferable that the mammary prosthesis of the present invention includes the adhesive layer since it is possible to improve an adhesive force of the mammary prosthesis of the present invention with respect to the human body.

In addition, in the mammary prosthesis of the present invention, the through hole does not necessarily have to be formed. However, it is preferable that the through hole is formed since through the through hole, it is possible to introduce a gas or a liquid into the hollow space formed inside the first layer from the exterior of the mammary prosthesis of the present invention and retain the introduced gas or liquid in the hollow space.

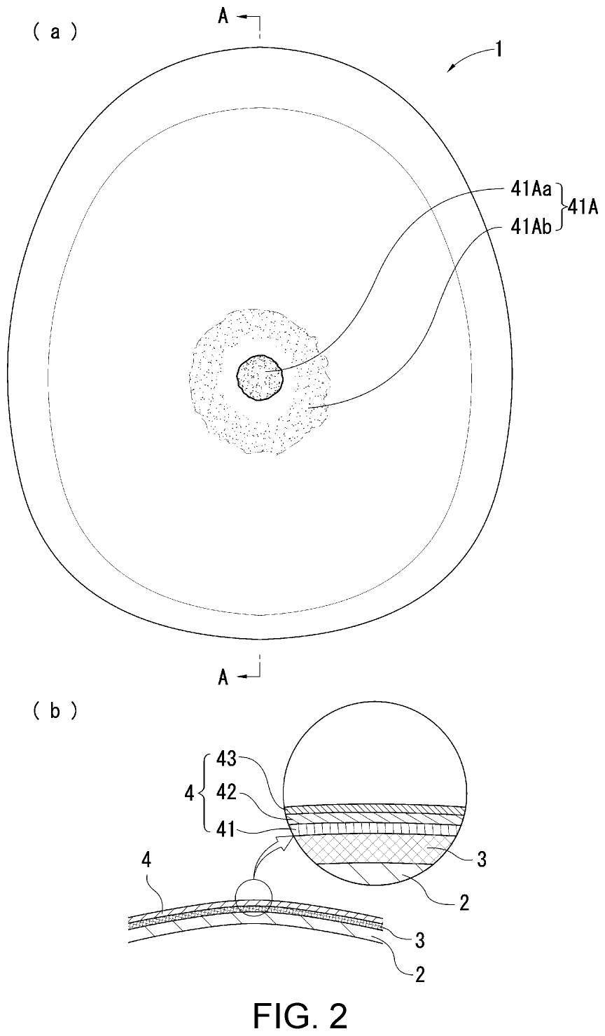

FIG. 2(a) is a schematic plan view of a second embodiment of the mammary prosthesis to which the present invention is applied, and FIG. 2(b) is a schematic partial sectional view of the vicinity of a nipple region when the mammary prosthesis shown in FIG. 2(a) is cut along line A-A.

A mammary prosthesis 1 of the second embodiment of the present invention shown in FIG. 2 is different from the mammary prosthesis 1 of the first embodiment of the present invention shown in FIG. 1 in a point that a nipple portion is not provided in a first layer 2, a colored layer 41 has a nipple region 41A in which a nipple is displayed, and the nipple region 41A and other regions of the colored layer 41 are positioned in substantially the same plane. It is noted that the mammary prosthesis of the second embodiment of the present invention is the same as the mammary prosthesis of the first embodiment of the present invention in other points.

The nipple region 41A is configured by a papilla region 41Aa and an areola region 41Ab shown around the papilla region 41Aa.

As shown in FIG. 2, in the mammary prosthesis 1 of the second embodiment of the present invention, the nipple region 41A and other regions of the colored layer 41 are positioned in substantially the same plane, and the nipple region 41A does not protrude. Thus, by putting on the mammary prosthesis 1 of the second embodiment of the present invention, it is possible to make the nipple inconspicuous even when the nipple is seen through clothing.

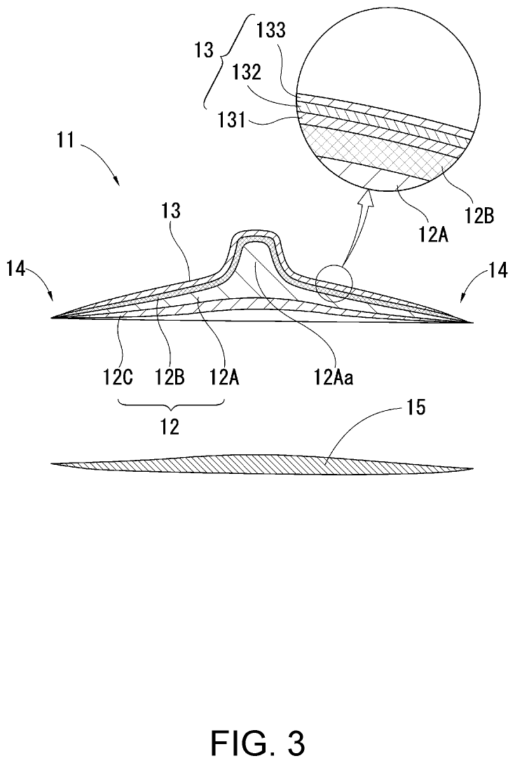

FIG. 3 is a schematic sectional view showing an example of an artificial nipple to which the present invention is applied. An artificial nipple 11 of the present invention shown in FIG. 3 includes a main body layer 12. The main body layer 12 is configured by a first main body configuration layer 12A, a second main body configuration layer 12B, and a third main body configuration layer 12C.

In addition, the first main body configuration layer 12A is configured by containing a mixture of silicone rubber and at least two types of thickeners whose viscosities are different from each other. In addition, a protruding papilla portion 12Aa is integrally provided in part of the first main body configuration layer 12A on one side.

In addition, softness of the first main body configuration layer 12A having such a configuration is softer than softness of a silicone rubber layer not containing such thickeners, and the softness is kept at least for 2 years.

In addition, various silicone rubbers can be used as the silicone rubber used for the first main body configuration layer 12A. However, specifically, for example, it is possible to use the same silicone rubber as the silicone rubber used for the first layer 2 which is provided in the mammary prosthesis 1 of the present invention. That is, as the silicone rubber used for the first main body configuration layer 12A, specifically, an example includes silicone rubber having a viscosity of 1,500 to 6,000 mPa·s at a normal temperature.

In addition, the thickeners used for the first main body configuration layer 12A are not particularly limited as long as the thickeners are at least two types of thickeners whose viscosities are different from each other.

Specifically, for example, it is possible to use the same thickeners as the thickeners used for the first layer 2 which is provided in the mammary prosthesis 1 of the present invention. That is, as the thickeners used for the first main body configuration layer 12A, specifically, an example includes at least two types of thickeners whose viscosities are different from each other selected from a group consisting of thickeners which have a viscosity of 100 to 2,500 mPa·s at a normal temperature.

In addition, the second main body configuration layer 12B is configured by containing silicone rubber. In addition, the second main body configuration layer 12B is arranged on the outer side of the first main body configuration layer 12A, and is a layer harder than the first main body configuration layer 12A. That is, the second main body configuration layer 12B is laminated on the first main body configuration layer 12A on one side where the papilla portion 12Aa is provided.

In addition, the third main body configuration layer 12C is configured by containing silicone rubber. In addition, the third main body configuration layer 12C is arranged on the inner side of the first main body configuration layer 12A, and is a layer harder than the first main body configuration layer 12A.

That is, the third main body configuration layer 12C is laminated on the first main body configuration layer 12A on the opposite side of the one side where the papilla portion 12Aa is provided.

In addition, although the silicone rubber contained in the second main body configuration layer 12B and the silicone rubber contained in the third main body configuration layer 12C are the same as each other, the silicone rubbers may be different from each other.

In addition, the main body layer 12 is attachable to a silicone rubber layer configured by containing silicone rubber, for example, a mammary prosthesis.

By the main body layer 12 attachable to the silicone rubber layer configured by containing silicone rubber, it is possible to attach the artificial nipple 11 of the present invention to a hollow type mammary prosthesis inside which a hollow space is formed, or a filled type mammary prosthesis inside which no hollow space is formed and silicone rubber exists without any space.

In addition, it is also possible to attach the artificial nipple 11 of the present invention to the human body.

In addition, the artificial nipple 11 of the present invention includes an adhesive layer 15. The adhesive layer 15 is configured by containing silicone gel, and arranged attachably to and detachably from the third main body configuration layer 12C which is arranged on the outermost side of the main body layer 12.

In addition, in a case where the artificial nipple 11 of the present invention is attached to the human body, it is possible to provide the adhesive layer 15 in the artificial nipple 11 of the present invention. In addition, in a case where the artificial nipple 11 of the present invention is attached to a mammary prosthesis, it is possible to provide no adhesive layer 15 in the artificial nipple 11 of the present invention, and use an adhesive, etc., for almost permanently attaching the artificial nipple 11 of the present invention.

In addition, the artificial nipple 11 of the present invention includes an outer covering layer 13. The outer covering layer 13 is arranged on the outer side of the main body layer 12. That is, the outer covering layer 13 is laminated on the second main body configuration layer 12B on the opposite side of the first main body configuration layer 12A. In addition, the part of the second main body configuration layer 12B on which the outer covering layer 13 is laminated is a part to be positioned on the front surface side when the artificial nipple 11 of the present invention is attached to the mammary prosthesis.

In addition, the outer covering layer 13 is configured by a colored layer 131, a cover layer 132, and a reflection suppression layer 133.

The colored layer 131 is configured by containing a colorant, specifically, for example, a pigment. In addition, the colored layer 131 is laminated on the second main body configuration layer 12B on the opposite side of the first main body configuration layer 12A.

In addition, the cover layer 132 is capable of suppressing peeling of the colored layer 131. In addition, the cover layer 132 is arranged on the outer side of the colored layer 131. That is, the cover layer 132 is laminated on the colored layer 131 on the opposite side of the second main body configuration layer 12B.

In addition, the reflection suppression layer 133 is arranged on the outer side of the cover layer 132. That is, the reflection suppression layer 133 is laminated on the cover layer 132 on the opposite side of the colored layer 131.

In addition, the artificial nipple 11 of the present invention includes a thin skin portion 14. The thin skin portion 14 is configured by an edge portion of the first main body configuration layer 12A, an edge portion of the second main body configuration layer 12B, and an edge portion of the third main body configuration layer 12C, and is so thin that it becomes transparent or semi-transparent.

In addition, the thin skin portion 14 is provided integrally with the main body layer 12. However, it is also possible to attach the thin skin portion 14 to an edge portion of the main body layer 12 as a separate body from the main body layer 12.

In addition, the main body layer 12 provided in the artificial nipple 11 of the present invention has a substantially flat shape as shown in FIG. 3, excluding the papilla portion 12Aa. However, the main body layer 12 can also have, for example, a curved shape in a mound to the side where the papilla portion 12Aa is provided.

In addition, the artificial nipple of the present invention does not necessarily have to include the adhesive layer. However, it is preferable that the artificial nipple of the present invention includes the adhesive layer since it is possible to improve an adhesive force of the artificial nipple of the present invention with respect to the human body.

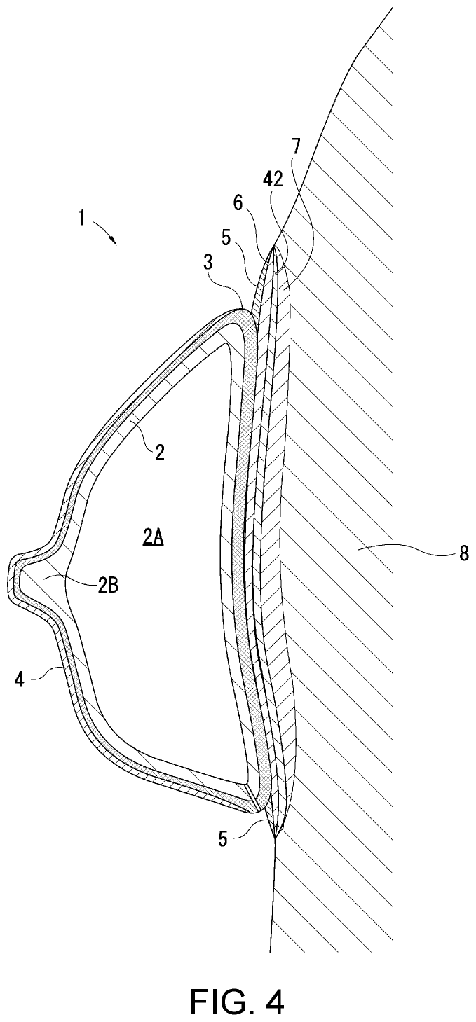

FIG. 4 is a schematic sectional view of the mammary prosthesis of the first embodiment to which the present invention is applied, the mammary prosthesis being put on a chest portion of a human body. As shown in FIG. 4, the mammary prosthesis 1 of the first embodiment of the present invention is put on a chest portion 8 in a state where the adhesive layer 7 is sandwiched between the polyethylene sheet layer 6 and the chest portion 8 of the human body.

In addition, when the mammary prosthesis 1 of the first embodiment of the present invention is detached from the chest portion 8, for example, the thin skin portion 5 of the mammary prosthesis 1 of the present invention is pinched by fingers and the mammary prosthesis 1 of the present invention is pulled apart while slightly compressing the mammary prosthesis 1 of the present invention and letting air in between the adhesive layer 7 and the chest portion 8.

### Example 1

First silicone rubber (trade name: VST-05, manufactured by Factor II, Incorporated) and a curing agent (trade name: VST-05-C, manufactured by Factor II, Incorporated) were mixed, and a first silicone rubber mixture was obtained.

In addition, a first thickener having a viscosity of 300 mPa·s and containing a polyether compound, and a second thickener having a viscosity of 1,800 mPa·s and containing a silicone polyether copolymer were added to the first silicone rubber mixture.

In addition, second silicone rubber (trade name: VST-50, manufactured by Factor II, Incorporated) and a curing agent (trade name: VST-50-C, manufactured by Factor II, Incorporated) were mixed, and a second silicone rubber mixture was obtained.

In order to form the second layer 3 of the mammary prosthesis 1 of the present invention, the second silicone rubber mixture was applied to a first mammary prosthesis formation mold in which a recessed part formed in a shape corresponding to an external form of the mammary prosthesis which includes the nipple portion and a peripheral edge part positioned around the recessed part are provided.

In addition, in order to form the second layer 3 of the mammary prosthesis 1 of the present invention, the second silicone rubber mixture was also applied to a second mammary prosthesis formation mold in which a raised portion is provided.

Next, in order to form the first layer 2 of the mammary prosthesis 1 of the present invention, the first silicone rubber mixture to which the first thickener and the second thickener were added was applied to the second silicone rubber mixture applied to the first mammary prosthesis formation mold.

In addition, in order to form the first layer 2 of the mammary prosthesis 1 of the present invention, the first silicone rubber mixture to which the first thickener and the second thickener were added was also applied to the second silicone rubber mixture applied to the second mammary prosthesis formation mold.

Next, the first mammary prosthesis formation mold and the second mammary prosthesis formation mold were attached to each other while letting the points to which the silicone rubbers were applied face each other.

At this time, a clearance exists between the recessed part of the first mammary prosthesis formation mold and the raised part of the second mammary prosthesis formation mold. This clearance becomes the hollow space inside the first layer 2 of the mammary prosthesis 1 of the present invention.

After at least 12 hours passed after the close attachment, the first mammary prosthesis formation mold and the second mammary prosthesis formation mold in an attached state were cured within a temperature range from room temperature to 70° C.

After the curing was finished, the second mammary prosthesis formation mold was pulled apart from the first mammary prosthesis formation mold, and a silicone rubber molded product was left in the first mammary prosthesis formation mold.

By a peripheral edge part where the first mammary prosthesis formation mold and the second mammary prosthesis formation mold were attached to each other closely, a thin skin part of the peripheral edge portion of the mammary prosthesis 1 of the present invention was formed. However, after all the thin skin part was cut off, in order to form the thin skin portion 5 of the mammary prosthesis 1 of the present invention, the second silicone rubber mixture was thinly applied to the peripheral edge part of the first mammary prosthesis formation mold.

The first mammary prosthesis formation mold was cured within the temperature range from room temperature to 70° C.

Next, in order to form the polyethylene sheet layer 6, a polyethylene sheet having a thickness of 15 μm was arranged on the silicon molded product of the first mammary prosthesis formation mold.

In order to form the cover layer 42, a mixture of the silicone rubber (trade name: VST-50, manufactured by Factor II, Incorporated) and the curing agent (trade name: VST-50-C, manufactured by Factor II, Incorporated) was applied to the polyethylene sheet.

Meanwhile, a first silicone gel material (trade name: GEL-GUM10(A), manufactured by TOMITA MATEQS) and a second silicone gel material (trade name: GEL-GUM10(B), manufactured by TOMITA MATEQS) were mixed, and a silicone gel material mixture was obtained. The obtained silicone gel material mixture and the silicone rubber (trade name: VST-05, manufactured by Factor II, Incorporated) were mixed by a mass ratio of 10:1, and a silicone mixture was obtained.

Next, in order to form the adhesive layer 7, the silicone mixture was applied to the mixture of the silicone rubber and the curing agent applied to the polyethylene sheet, and the first mammary prosthesis formation mold was cured within the temperature range from room temperature to 70° C.

The first mammary prosthesis formation mold was removed. The mammary prosthesis 1 of the present invention at this time point includes the first layer 2 inside which the hollow space 2A is formed, and the second layer 3 laminated on the outer side part of the first layer 2 in a state of covering the entire first layer 2. In addition, the protruding nipple portion 2B is integrally provided in part of the curved part of the first layer 2 on the opposite side of the hollow space 2A.

In addition, the mammary prosthesis 1 of the present invention at this time point further includes the thin skin portion 5 attached to the second layer 3 and provided over the entire peripheral edge portion, and the polyethylene sheet layer 6 laminated on the substantially flat part of the second layer 3 on the opposite side of the first layer 2.

Further, the mammary prosthesis 1 of the present invention at this time point includes the cover layer 42 laminated on the polyethylene sheet layer 6, and the adhesive layer 7 laminated on the polyethylene sheet layer 6 via the cover layer 42.

In addition, when hardness of the first layer 2 immediately after formation of the first layer 2 was measured by a rubber hardness meter, “Type A Durometer,” the hardness was “28.” In addition, when hardness of the second layer 3 immediately after formation of the second layer 3 was measured by the rubber hardness meter, “Type A Durometer,” the hardness was “31.”

In addition, when the hardness of the first layer 2 was measured again by the rubber hardness meter, “Type A Durometer” after about 2 years after the formation of the first layer 2, the hardness was “29” and it was confirmed that its softness was kept.

In addition, even when the obtained mammary prosthesis 1 of the present invention was deformed by applying forces from various angles, unnatural wrinkles were not made.

Next, by using a brush, a colorant was applied to the curved part of the second layer 3 on the opposite side of the first layer 2. The mammary prosthesis 1 of the present invention to which the colorant was applied was cured within the temperature range from room temperature to 70° C., and the colored layer 41 was formed.

The mixture of the silicone rubber (trade name: VST-50, manufactured by Factor II, Incorporated) and the curing agent (trade name: VST-50-C, manufactured by Factor II, Incorporated) was applied to the colored layer 41 on the opposite side of the second layer 3. After the application, the mammary prosthesis 1 of the present invention was cured within the temperature range from room temperature to 70° C., and the cover layer 42 was formed.

Further, a mixture of an acetoxy dispersing agent containing cyclohexane (product code: MD-564, manufactured by Factor II, Incorporated) and a silicone adhesive (trade name: A-564, manufactured by Factor II, Incorporated) was applied to the cover layer 42 on the opposite side of the colored layer 41. After the application, the mammary prosthesis 1 of the present invention was cured within the temperature range from room temperature to 70° C., and the reflection suppression layer 43 was formed.

The texture of the obtained mammary prosthesis 1 of the present invention becomes closer to the texture of the human skin by the external coloring based on the colored layer 41. In addition, by the cover layer 42, the texture brought close to the texture of the human skin is easily maintained. Further, by the reflection suppression layer 43, the reflection of light on the surface of the mammary prosthesis 1 of the present invention is suppressed, and the texture becomes even closer to the texture of the human skin.

### Example 2

First silicone rubber (trade name: VST-05, manufactured by Factor II, Incorporated) and a curing agent (trade name: VST-05-C, manufactured by Factor II, Incorporated) were mixed, and a first silicone rubber mixture was obtained.

Further, a first thickener having a viscosity of 300 mPa·s and containing a polyether compound, and a second thickener having a viscosity of 1,800 mPa·s and containing a silicone polyether copolymer were added to the first silicone rubber mixture.

In addition, second silicone rubber (trade name: VST-50, manufactured by Factor II, Incorporated) and a curing agent (trade name: VST-50-C, manufactured by Factor II, Incorporated) were mixed, and a second silicone rubber mixture was obtained.

In order to form the second main body configuration layer 12B of the main body layer 12 of the artificial nipple 11 of the present invention, the second silicone rubber mixture was applied to an artificial nipple formation mold in which a recessed part formed in a shape corresponding to an external form of the artificial nipple which includes the papilla portion and a peripheral edge part positioned around the recessed part are provided.

Next, in order to form the first main body configuration layer 12A of the main body layer 12 of the artificial nipple 11 of the present invention, the first silicone rubber mixture to which the first thickener and the second thickener were added was applied to the second silicone rubber mixture applied to the artificial nipple formation mold.

In addition, in order to form the third main body configuration layer 12C of the main body layer 12 of the artificial nipple 11 of the present invention, the second silicone rubber mixture was applied to the first silicone rubber mixture to which the first thickener and the second thickener were added.

Next, as for the artificial nipple formation mold to which the first silicone rubber mixture added with the first thickener and the second thickener and the second silicone rubber mixture were applied, the silicone rubber mixture was cured within a temperature range from room temperature to 70° C.

Meanwhile, a first silicone gel material (trade name: GEL-GUM10(A), manufactured by TOMITA MATEQS) and a second silicone gel material (trade name: GEL-GUM10(B), manufactured by TOMITA MATEQS) were mixed, and a silicone gel material mixture was obtained. The obtained silicone gel material mixture and the silicone rubber (trade name: VST-05, manufactured by Factor II, Incorporated) were mixed by a mass ratio of 10:1, and a silicone mixture was obtained.

Next, in order to form the adhesive layer 15, the silicone mixture was applied to the third main body configuration layer 12C of the main body layer 12, and the artificial nipple formation mold was cured within the temperature range from room temperature to 70° C.

After the curing was finished, the artificial nipple formation mold was removed. The artificial nipple 11 of the present invention at this time point includes the main body layer 12, and the main body layer 12 is configured by the first main body configuration layer 12A, the second main body configuration layer 12B, and the third main body configuration layer 12C.

That is, the protruding papilla portion 12Aa is integrally provided in part of the first main body configuration layer 12A on one side. In addition, the second main body configuration layer 12B is laminated on the first main body configuration layer 12A on one side where the papilla portion 12Aa is provided. Further, the third main body configuration layer 12C is laminated on the first main body configuration layer 12A on the opposite side of the one side where the papilla portion 12Aa is provided.

In addition, when hardness of the main body layer 12 immediately after formation of the main body layer 12 was measured by a rubber hardness meter, “Type A Durometer,” the hardness was “27.” In addition, when the hardness of the main body layer 12 was measured again by the rubber hardness meter, “Type A Durometer” after about 2 years after the formation of the main body layer 12, the hardness was “28” and it was confirmed that its softness was kept.

Next, by using a brush, a colorant was applied to the second main body configuration layer 12B on the opposite side of the first main body configuration layer 12A. The artificial nipple 11 of the present invention to which the colorant was applied was cured within the temperature range from room temperature to 70° C., and the colored layer 131 was formed.

The mixture of the silicone rubber (trade name: VST-50, manufactured by Factor II, Incorporated) and the curing agent (trade name: VST-50-C, manufactured by Factor II, Incorporated) was applied to the colored layer 131 on the opposite side of the second main body configuration layer 12B. After the application, the artificial nipple 11 of the present invention was cured within the temperature range from room temperature to 70° C., and the cover layer 132 was formed.

Further, the mixture of the acetoxy dispersing agent containing cyclohexane (product code: MD-564, manufactured by Factor II, Incorporated) and the silicone adhesive (trade name: A-564, manufactured by Factor II, Incorporated) was applied to the cover layer 132 on the opposite side of the colored layer 131. After the application, the artificial nipple 11 of the present invention was cured within the temperature range from room temperature to 70° C., and the reflection suppression layer 133 was formed.

The texture of the obtained artificial nipple 11 of the present invention becomes closer to the texture of the human skin by the external coloring based on the colored layer 131. In addition, by the cover layer 132, the texture brought close to the texture of the human skin is easily maintained. Further, by the reflection suppression layer 133, the reflection of light on the surface of the artificial nipple 11 of the present invention is suppressed, and the texture becomes even closer to the texture of the human skin.

As described above, the mammary prosthesis of the present invention includes the first layer configured by containing the mixture of silicone rubber and at least two types of thickeners whose viscosities are different from each other. Thus, the mammary prosthesis is softer than a mammary prosthesis not containing such thickeners, and it is possible to keep its softness for a long time.

In addition, with the first layer configured by containing the mixture of silicone rubber and at least two types of thickeners whose viscosities are different from each other and the second layer arranged on the outer side of the first layer, the second layer being harder than the first layer, the mammary prosthesis of the present invention becomes harder stepwise from the inner side to the outer side. Thus, even when a force is applied from the exterior, unnatural wrinkles are not easily made.

Therefore, the mammary prosthesis of the present invention is less likely to become unnatural in comparison to a human breast.

In addition, due to the softness of the mammary prosthesis of the present invention, it is possible to flexibly change a shape of the mammary prosthesis even in a brassiere and to create cleavage.

In addition, the artificial nipple of the present invention also includes the main body layer configured by containing the mixture of silicone rubber and at least two types of thickeners whose viscosities are different from each other. Thus, the artificial nipple is softer than an artificial nipple not containing such thickeners, and it is possible to keep its softness for a long time.

In addition, the artificial nipple of the present invention includes the colored layer. This colored layer is arranged on the outer side of the main body layer and configured by containing the colorant. Thus, it is possible to perform the external coloring and bring the texture closer to the texture of the human skin than in the case of internal coloring where silicone rubber contains a colorant.

Further, the artificial nipple of the present invention includes the cover layer. This cover layer is arranged on the outer side of the colored layer and is capable of suppressing the peeling of the colored layer. Thus, it is possible to maintain the texture brought close to the texture of the human skin.

In addition, the artificial nipple of the present invention includes the reflection suppression layer. This reflection suppression layer is arranged on the outer side of the cover layer and is capable of suppressing the reflection of light of the cover layer or the colored layer. Thus, it is possible to bring the texture even closer to the texture of the human skin.

Therefore, the artificial nipple of the present invention is less likely to become unnatural in comparison to a human nipple particularly in terms of appearance.

### DESCRIPTION OF REFERENCE NUMERALS

1 mammary prosthesis
2 first layer
2A hollow space
2B nipple portion
3 second layer
4 outer covering layer
41 colored layer
41A nipple region
41Aa papilla region
41Ab areola region
42 cover layer
43 reflection suppression layer
5 thin skin portion
6 polyethylene sheet layer
7 adhesive layer
8 chest portion
9 through hole
11 artificial nipple
12 main body layer
12A first main body configuration layer
12Aa papilla portion
12B second main body configuration layer
12C third main body configuration layer
13 outer covering layer
131 colored layer
132 cover layer
133 reflection suppression layer
14 thin skin portion
15 adhesive layer

1. A mammary prosthesis, comprising:
a first layer configured by containing a mixture of silicone rubber and at least two types of thickeners whose viscosities are different from each other, the first layer inside which a hollow space is formed; and
a second layer arranged on an outer side of the first layer, the second layer being harder than the first layer, the second layer being configured by containing silicone rubber.

2. The mammary prosthesis according to claim 1, comprising:
a colored layer arranged on an outer side of the second layer, the colored layer being configured by containing a colorant; and
a cover layer arranged on an outer side of the colored layer, the cover layer being capable of suppressing peeling of the colored layer.

3. The mammary prosthesis according to claim 2, wherein the colored layer has a nipple region in which a nipple is displayed, and the nipple region and other regions of the colored layer are positioned in the same plane.

4. The mammary prosthesis according to claim 2, comprising:
a reflection suppression layer arranged on an outer side of the cover layer, the reflection suppression layer being capable of suppressing reflection of light of the cover layer or the colored layer.

5. The mammary prosthesis according to claim 1, comprising:
a thin film layer arranged on an outer side of the second layer, the thin film layer being capable of blocking a substance, with the thin film layer being transparent.

6. The mammary prosthesis according to claim 5, comprising:
an adhesive layer arranged attachably to and detachably from a layer which is arranged on the outermost side, the adhesive layer being configured by containing an adhesive material.

7. The mammary prosthesis according to claim 1, wherein a through hole passing through the first layer and the second layer and providing communication between an exterior and the hollow space, the through hole having such a size that a gas or a liquid in the hollow space is not easily leaked out to the exterior is formed in the first layer and the second layer.

8. A mammary prosthesis, comprising:
a silicone rubber layer configured by containing silicone rubber, the silicone rubber layer inside which a hollow space is formed;
a nipple portion provided in part of the silicone rubber layer on an opposite side of the hollow space, the nipple portion being configured by containing a mixture of silicone rubber and at least two types of thickeners whose viscosities are different from each other;
a colored layer arranged on an outer side of the silicone rubber layer and on an outer side of the nipple portion, the colored layer being configured by containing a colorant;
a cover layer arranged on an outer side of the colored layer, the cover layer being capable of suppressing peeling of the colored layer; and
a reflection suppression layer arranged on an outer side of the cover layer, the reflection suppression layer being capable of suppressing reflection of light of the cover layer or the colored layer.

9. An artificial nipple, comprising:
a main body layer configured by containing a mixture of silicone rubber and at least two types of thickeners whose viscosities are different from each other;
a colored layer arranged on an outer side of the main body layer, the colored layer being configured by containing a colorant;
a cover layer arranged on an outer side of the colored layer, the cover layer being capable of suppressing peeling of the colored layer; and
a reflection suppression layer arranged on an outer side of the cover layer, the reflection suppression layer being capable of suppressing reflection of light of the cover layer or the colored layer.
